(19) United States
(12) Patent Application Publication
(10) Pub. No.: US 2008/0319196 A1
(43) Pub. Date: Dec. 25, 2008

(54) Process for Synthesizing Remifentanil

(76) Inventor: Brian K. Cheng, Chesterfield, MO (US)

Correspondence Address: Mallinckrodt Inc., 675 McDonnell Boulevard, HAZELWOOD, MO, 63042, US

(21) Appl. No.: 12/093,517

(22) PCT Filed: Oct. 23, 2006
(86) PCT No.: PCT/US06/41312

§ 371 Date: May 13, 2008

Related U.S. Application Data
(60) Provisional application No. 60/737,655, filed on Nov. 17, 2005.

(51) Int. Cl. C07D 211/56

(52) U.S. Cl. 546/224

(57) ABSTRACT

An improved process for synthesizing opiate or opioid analgesics and anesthetics, and intermediates thereof is provided. In particular, processes of synthesizing intermediates for use in the preparation of synthetic opiate or opioid compounds such as, for example, remifentanil, carfentanil, sufentanil, fentanyl, and alfentanil are disclosed. The preparation process requires fewer steps, and results in reduced costs and higher efficiency than processes known in the art for producing remifentanil and carfentanil.

### FIELD OF THE INVENTION

The present invention generally relates to a process for synthesizing opiate or opioid analgesics and anesthetics, and precursors thereof. In particular, the present invention relates to processes of synthesizing intermediates for use in the preparation of synthetic opiate or opioid compounds such as, for example, remifentanil, carfentanil, sufentanil, fentanyl, and alfentanil. In particular, the present invention relates to a preparation process that requires fewer steps, reduced costs, and higher efficiency than processes known in the art for producing remifentanil and carfentanil.

### BACKGROUND OF THE INVENTION

Analgesics, such as remifentanil and carfentanil, have been prepared in synthetic processes comprising six and seven steps. Examples of such processes are outlined in U.S. Pat. Nos. 5,106,983 and 5,019,583. However, these syntheses often require multiple protection and deprotection steps of reactive moieties, resulting in increased process costs due to reduced production efficiency and additional material costs.

A process with fewer process steps would be beneficial in improving process efficiencies and reducing the cost of synthesizing analgesics.

### SUMMARY OF THE INVENTION

Among the several features of the present invention, therefore, can be noted the provision of a process for synthesizing intermediates for use in the preparation of synthetic opiate or opioid compounds such as, for example, remifentanil, carfentanil, sufentanil, fentanyl, and alfentanil; the provision of preparing an analgesic; the provision of a process that requires fewer steps for synthesizing remifentanil; the provision of a process that requires fewer steps for synthesizing carfentanil; with an alkylating compound in the presence of a solvent to form intermediate compound (V):

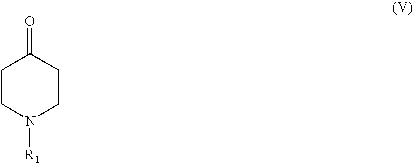

wherein R₁ is a hydrocarbyl or substituted hydrocarbyl. Reacting the intermediate compound (V) with an amine and a cyanide compound, in the presence of a first acid to form an intermediate compound (VI):

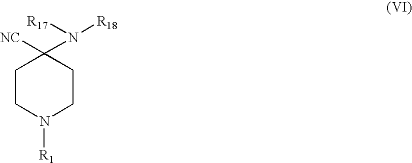

wherein R₁₇ and R₁₈ are independently selected from the group comprising hydrogen, hydrocarbyl, and substituted hydrocarbyl. Reacting the intermediate compound (VI) with a second acid to form an intermediate amide. Reacting the intermediate amide with an alcohol, R₁₉OH, to form an intermediate compound (VII):

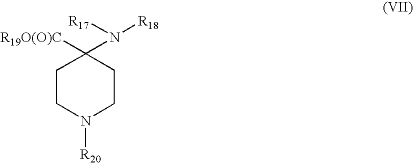

wherein R₁₉ is hydrocarbyl or substituted hydrocarbyl and R₂₀ is hydrocarbyl or substituted hydrocarbyl. Reacting the intermediate compound (VII) with an acylating agent to form a compound (VIII) having the formula:

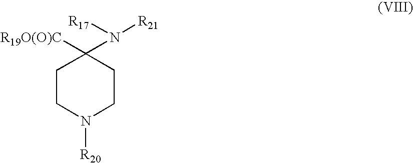

wherein R₂₁ is —C(O)—R₂₂, wherein R₂₂ is hydrocarbyl or substituted hydrocarbyl.

In another aspect, the invention is directed to a process for synthesizing an intermediate of opiate or opioid analgesics or anesthetics. The process comprises reacting compound (IV) having the formula:

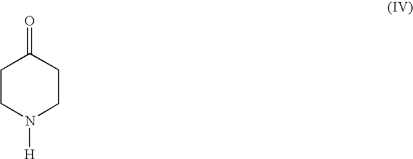

with an alkylating agent, a solvent, and a base to form an intermediate compound (V) having the formula:

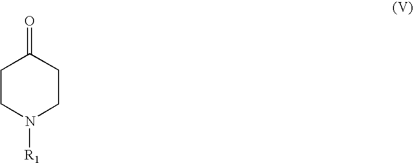

wherein R₁ is hydrocarbyl or substituted hydrocarbyl.

In another aspect, the invention is directed to a process for synthesizing an intermediate of opiate or opioid analgesics or anesthetics. The process comprises reacting intermediate compound (V) having the formula:

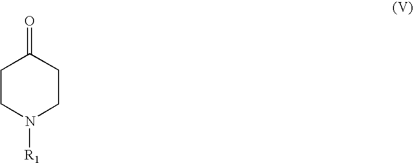

with cyanide compound, an amine, and an acid to form an intermediate compound (VI) having the formula:

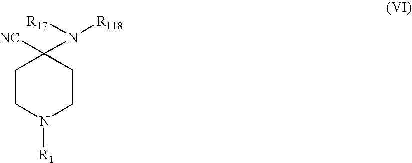

wherein R₁ is a hydrocarbyl or substituted hydrocarbyl; and R₁₇ and R₁₈ are independently selected from hydrogen, hydrocarbyl, or substituted hydrocarbyl.

In another aspect, the present invention is directed to a process of synthesizing an intermediate of opiate or opioid analgesics or anesthetics comprising reacting intermediate compound (VI) having the formula:

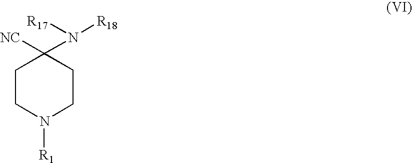

with an acid and an alcohol, R₁₉OH, in a reaction mixture to form an intermediate compound (VII) having the formula:

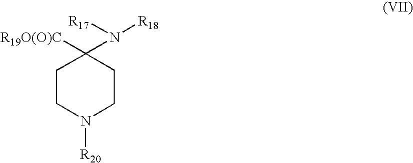

wherein R₁ is a hydrocarbyl or substituted hydrocarbyl; R₁₇ and R₁₈ are independently selected from hydrogen, hydrocarbyl, or substituted hydrocarbyl; R₁₉ is hydrocarbyl or substituted hydrocarbyl; and R₂₀ is hydrocarbyl or substituted hydrocarbyl. Other aspects and features of this invention will be in part apparent and in part pointed out hereinafter.

### DETAILED DESCRIPTION

In accordance with the present invention, an improved process for synthesizing analgesics has been discovered. The improved process reduces the process steps required to synthesize the analgesics. The process also improves yield of synthesized analgesic product as compared to processes known in the art.

In one embodiment, the process of the present invention results in the synthesis of a compound having the formula (I):

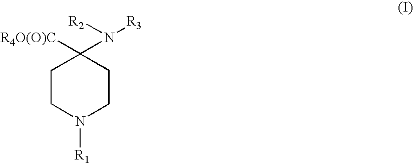

wherein R₁ is hydrocarbyl or substituted hydrocarbyl, R₂ and R₃ are independently hydrogen, hydrocarbyl or substituted hydrocarbyl, and R₄ is hydrocarbyl or substituted hydrocarbyl.

In another embodiment, R₁ is hydrocarbyl or substituted hydrocarbyl, R₂ is a phenyl or substituted phenyl, R₃ is hydrogen, hydrocarbyl or substituted hydrocarbyl, and R₄ is hydrocarbyl or substituted hydrocarbyl. In one example, R₂ is a phenyl substituted with one or more halo, silicon, boron, nitrogen, or oxygen atoms.

In one embodiment, the present invention can be used to synthesize remifentanil, chemically identified as 3-[4-methoxycarbonyl-4-[(1-oxopropyl)phenylamino]-1-piperidine]propanoic acid methyl ester, having the formula (II), utilizing a piperidone starting material.

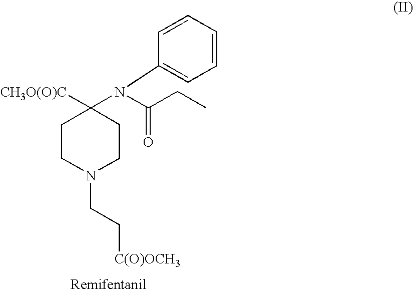

In another embodiment, the present invention can be used to synthesize carfentanil, chemically identified as 4((1-oxopropyl)phenylamino)-1-(2-phenylethyl)-4-piperidinecarboxylic acid, methyl ester, having the formula (III), by utilizing either a piperidone or a 1-(2-phenylethyl)-4-piperidone starting material.

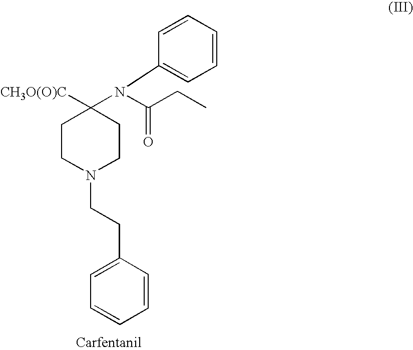

The improved process of the present invention for synthesizing opiate or opioid analgesics and anesthetics includes the synthesis of a series of several novel intermediates. Scheme 1, below, illustrates a first step in the process wherein 4-piperidone hydrochloride, compound (IV) is alkylated to form intermediate compound (V).

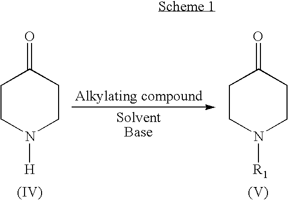

In one embodiment, an acid salt of compound (IV), for example 4-piperidone hydrochloride, is mixed in a reaction mixture with an alkylating agent in Step 1 in the presence of a solvent and a base to form intermediate compound (V), wherein R₁ is hydrocarbyl or substituted hydrocarbyl. Preferably, R₁ is a group selected from R₅OC(O)R₆—, R₇C(O)OR₈—, R₉OR₁₀OC(O)R₁₁—, R₁₂R₁₃—, and R₁₄R₁₅—, wherein R₅, R₆, R₇, R₈, R₉, R₁₀, R₁₁, R₁₃, and R₁₅ are hydrocarbyl or substituted hydrocarbyl, R₁₂ is cycloalkyl, and R₁₄ is a heterocyclic comprising 1 to 5 hetero-atoms. Preferably, R₅, R₆, R₇, R₈, R₉, R₁₀, R₁₁, R₁₃, and R₁₅ are alkyl, alkoxy, alkenyl, and alkenyloxy groups, R₁₂ is a 5- to 7-member cycloalkyl, and R₁₄ is a 5- to 7-member heterocyclic; more preferably, R₅, R₆, R₇, R₈, R₉, R₁₀, R₁₁, R₁₃, and R₁₅ are linear or branched alkyl, alkoxy, alkenyl, and alkenyloxy groups having about 1 to about 18 carbon atoms, R₁₂ is a 5- to 7-member cycloalkyl, and R₁₄ is a 5- to 7-member heterocyclic comprising 1 to 5 hetero-atoms selected from oxygen, sulfur, and nitrogen; still more preferably, R₁ is methyl propionate, ethyl propionate, 2-phenylethyl, 2-(2-thienyl)ethyl, and 2-(4-ethyl-4,5-digydro-5-oxo-1H-tetrazol-1-yl)ethyl.

In one embodiment, the reaction mixture comprises about 1 molar equivalent to about 3 molar equivalents of alkylating agent and about 1 molar equivalent to about 3 molar equivalents of an acid scavenger (i.e., a base) to 1 molar equivalent of compound (IV). Preferably, the reaction mixture is charged with about 1 to about 1.5 equivalents of an alkylating agent and about 1 equivalent to about 1.5 equivalents of an acid scavenger to 1 equivalent of 4-piperidine hydrochloride. The solvent to compound (IV) ratio on a wt. basis is about 1:10 to 1:100.

The temperature of the reaction mixture during the reaction ranges from about −10° C. to about 65° C. In another embodiment, the reaction temperature ranges from about 10° C. to about 40° C. The reaction mixture is permitted to react up to a couple of days. In one example, the reaction is carried out up to about 24 hours. In another example, the reaction time is from about 2 hours to about 6 hours.

General examples of alkylating agents include compounds having the structure:

L-R₂₃—R₁₆

wherein L is a displacement or leaving group L. In one embodiment, L, R₁₆, and R₂₃ are hydrocarbyl or substituted hydrocarbyl. Preferably, L is a halide, toluenesulfonate, or methylsulfonate; R₂₃ is a hydrocarbyl or substituted hydrocarbyl group having 1 to 18 carbons; and R₁₆ is selected from R₅OC(O)—, R₇C(O)O—, R₉OR₁₀OC(O)—, R₁₂—, and R₁₄—, wherein R₅, R₇, R₉, R₁₁, R₁₂, and R₁₄, are as defined above; preferably, L is a halide, toluenesulfonate, or methylsulfonate, R₂₃ is ethyl, and R is —C(O)OCH₃, —C(O)OCH₂CH₃, -phenyl, -2-(2-thienyl), and -2-(4-ethyl-4,5-dihydro-5-oxo-1H-tetrazol-1-yl)ethyl.

The alkylating agents can also comprise an electron deficient moiety to an electron withdrawing group such as carbonyl, nitrile, carbonyl-oxy, alkyl carbonate, and alkyl-alkoxy carbonate. Some specific examples of the alkylating agents include methyl acrylate, ethyl acrylate, acrylic acid, acryronitrile, acrylamide, acrolein, phenylethyl halide, tolylate, mesoilate, styrene, and substituted styrene. An illustration of alkylating agents comprising an electron deficient moiety is as follows:

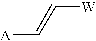

wherein A is hydrogen, hydrocarbyl, or substituted hydrocarbyl and W is hydrocarbyl, substituted hydrocarbyl, nitrile, and amide. In one example, A is hydrogen, an alkyl comprised of 1 to 18 carbons, aryl, substituted aryl, alkylaryl wherein the alkyl group is comprised of 1 to 18 carbons, and a hydrocarbyl or substituted hydrocarbyl 5- to 7-member ring; and W is carboxylic acid, carboxylic acid ester, nitrile, amide, carbonyl, or aryl.

The reaction mixture contains a base to neutralize the acid salt of compound (IV). In one embodiment, compound (IV) is the hydrochloride salt of 4-piperidone. Examples of the base include sodium hydroxide, potassium hydroxide, metal alkoxides, metal hydrides, metals, amines, quaternary alkyl ammonia hydroxides, and any other base in that can neutralize an acid salt of compound (IV). Examples of metal alkoxides and metal hydrides include sodium, potassium, cesium, magnesium, aluminum alkoxides and hydrides and the like. Examples of metals include scavenging metals such as sodium, potassium, magnesium, and the like.

The solvent used in the reaction mixture can include water and/or one or more organic solvents. Examples of organic solvents include, but are not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; alkanols, for example, methanol, ethanol, isopropanol, 1-butanol, tert-butanol, and the like; ketones such as 4-methyl-2-pentanone and the like; ethers such as 1,4-dioxane, tetrahydrofuran (THF), 1,1-oxybisethane, and the like; nitrobenzene; and mixtures thereof.

In one embodiment, compound V is isolated by quenching the reaction with water, crystallizing the product compound, and recovering compound V through filtration and drying. Compound V may be further purified through recrystallization with organic solvents.

In another embodiment, wherein compound V is a liquid, the compound V is isolated through solvent extraction and isolation procedures known in the art. Such isolation procedures can include evaporating solvent to recover the crude oil product. Depending on its physical properties, compound V is thereafter isolated by chromatography or distillation.

Scheme 2, below, illustrates a second step in the process of the present invention intermediate compound (VI) is synthesized.

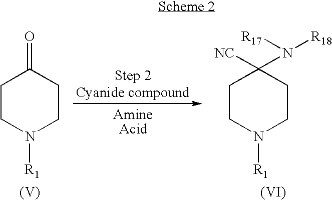

In Step 2, compound (V) is reacted with a cyanide compound and an amine in the presence of an acid in a reaction mixture to form compound (VI), wherein R₁₇ and R₁₈ are independently selected from hydrogen, hydrocarbyl, or substituted hydrocarbyl. Preferably, R₁₇ and R₁₈ are independently selected from hydrogen, alkyl, alkoxyalkyl, aryl, substituted aryl, and hydrocarbyl or substituted hydrocarbyl 5- to 7-member cyclic structure. In one example, R₁₇ and/or R₁₈ are independently a phenyl or substituted phenyl group.

In one embodiment, the reaction mixture comprises about 1 molar equivalent to about 3 molar equivalents of the amine and about 1 molar equivalent to about 3 molar equivalents of the cyanide compound to 1 molar equivalent of compound (V). The acidic medium to compound (V) ratio on a wt. basis is about 1:10 to 1:100.

In another embodiment, the reaction mixture is charged with about 1 molar equivalent to about 1.2 molar equivalents of the amine and about 1 molar equivalent to about 1.2 molar equivalents of the cyanide compound in a w/w ratio of about 10 to about 20 of an acidic medium.

The temperature of the reaction mixture during the reaction ranges from about −10° C. to about 65° C. In another example, the reaction temperature ranges from about 10° C. to about 40° C. The reaction mixture is permitted to react up to a couple of days. In one example, the reaction is carried out up to about 24 hours. In another example, the reaction time is from about 2 hours to about 6 hours.

Non-limiting examples of cyanide compounds include sodium cyanide, potassium cyanide, trimethylsilyl cyanide, hydrogen cyanide, and the like. Examples of the amine compounds utilized in Step 2 include alkyl amine, ammonia, and phenyl amine compounds. Examples of phenyl amine compounds include aniline and substituted phenyl amine compounds wherein the substituted constituents include hydrocarbyl or substituted hydrocarbyl groups having 1 to 18 carbons. The acid may include any organic or inorganic acid to adjust the pH below about 7. Non-limiting examples of acids include acetic acid, hydrochloric acid, sulfuric acid, phosphoric acid, oxalic acid, and the like. In one embodiment, acetic acid is utilized to adjust the reaction mixture pH to below about 7.

The reaction can be conducted in the presence or absence of water. If the reaction takes place under anhydrous conditions, excess amount of a solvent is used in the reaction mixture. In one embodiment, the solvent is comprised of organic solvents including, but not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; alcohols having one or more carbons, for example, methanol, ethanol, isopropanol, 1-butanol, tert-butanol, and the like; ketones such as 4-methyl-2-pentanone and the like; ethers such as 1,4-dioxane, tetrahydrofuran (THF), 11-oxybisethane, and the like; nitrobenzene; and mixtures thereof. In another embodiment, the solvent can contain between about 10% to about 99% acid. In another embodiment, the reaction mixture can contain up to about 90% water.

Compound (VI) can be isolated by utilizing isolation procedures known in the art such as those described for the above schemes.

In a third step of the present invention, intermediate compound (VII) is synthesized in a two-part step illustrated below in Scheme 3.

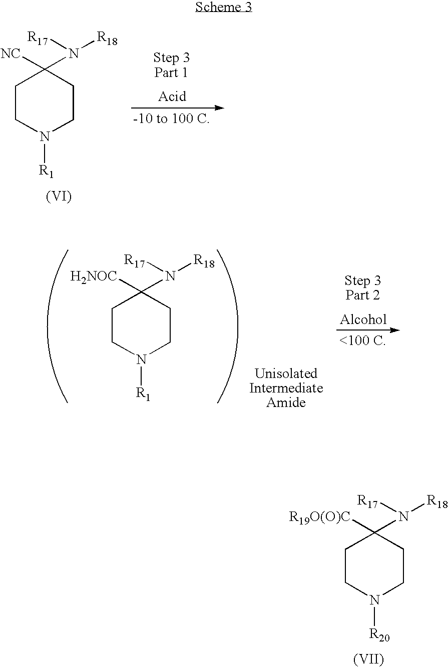

Step 3 is a two-part reaction taking place in a single reaction mixture wherein no product is isolated between the parts. In Part 1 of Step 3, compound (VI) is hydrolyzed with an acid and water to form an intermediate amide in situ. The reaction mixture can optionally comprise a solvent.

In one embodiment, the reaction mixture comprises about 3 molar equivalents to about 10 molar equivalents of the acid to 1 molar equivalent of compound (VI). In another embodiment, the reaction mixture comprises about 3 molar equivalents to about 5 molar equivalents of the acid to 1 molar equivalents of compound (VI).

In one embodiment, the reaction mixture temperature is from about −10° C. to about 40° C. In another example, the reaction mixture temperature is from about 15° C. to about 35° C. In still another example, the reaction mixture temperature is from about 10° C. to about 30° C. The reaction mixture is permitted to react up to a couple of days. In one example, the reaction is carried out up to about 24 hours. In another example, the reaction time is from about 2 hours to about 8 hours.

The acid source can be selected from organic or inorganic acids to adjust the pH of the reaction mixture below about 7. In one embodiment, the acid is selected from acetic acid, hydrochloric acid, sulfuric acid, methansulfonic acid, phosphoric acid, oxalic acid, and the like. In one example, the acid concentration is between 10% and about 99%, preferably between 70% and about 99%, with the balance comprising water. In still another example, the acid is selected from sulfuric acid or methansulfonic acid.

In one embodiment, the reaction mixture contains a solvent selected from the organic solvents described above for Scheme 2. In one example, the solvent comprises between about 10% to about 99% acid.

If the reaction takes place under anhydrous conditions, excess amount of alcohol is used as a solvent in the reaction mixture. In one embodiment, the alcohol is an aliphatic alcohol having 1 to 3 carbons.

In Part 2 of Step 3, an alcohol, R₁₉OH is added to the reaction mixture of Part 1 of Step 3, wherein R₁₉ is hydrocarbyl or substituted hydrocarbyl. The intermediate amide is esterified to form compound (VII), wherein R₁₉ is a hydrocarbyl or substituted hydrocarbyl corresponding to the alcohol used in the Part 2 of Step 3. R₂₀ is hydrocarbyl or substituted hydrocarbyl. In another example, R₂₀ is a group selected from R₅OC(O)R₆—, R₇C(O)OR₈—, R₉OR₁₀OC(O)R₁₁—, R₁₂R₁₃—, and R₁₄R₁₅—, wherein R₅, R₆, R₇, R₈, R₉, R₁₀, R₁₁, R₁₃, and R₁₁ are as defined above. When R₁ is an ester, the reaction transesterifies R₁ to R₂₀ to form the ester corresponding to the alcohol used in Part 2 of Step 3 (e.g., R₂₀ is transesterified to —OR₁₉).

In one embodiment, about 10 parts to about 50 parts of alcohol are added to the reaction mixture of Part 2 of Step 3. In one example, about 10 parts to about 20 parts of alcohol are added to the reaction mixture of Part 2 of Step 3.

In one embodiment, the reaction mixture temperature is from about −10° C. to about 75° C. In another example, the reaction mixture temperature is from about 40° C. to about 65° C. The reaction mixture is permitted to react for about 24 hours to about 150 hours. In another example, the reaction time is from about 60 hours to about 100 hours.

Examples of alcohols include, but are not limited to C₁-C₁₈ aliphatic alcohols, such as methanol, ethanol, propanol, isopropanol, butanol, tert-butanol, sec-butanol, pentanol, hexanol, aromatic alcohols, such as phenol, and the like. In one embodiment, the alcohol is selected from C₁-C₃ aliphatic alcohols.

Compound (VII) can be isolated by utilizing isolation procedures known in the art such as those described for the above schemes.

Finally, in a fourth step, intermediate compound (VIII) is synthesized as illustrated in Scheme 4:

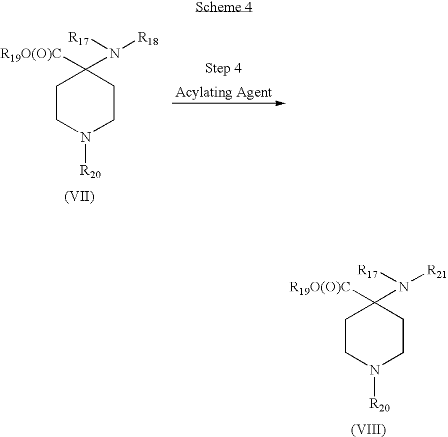

In Step 4, compound (VII) is reacted with an acylating agent in a reaction mixture containing a solvent to form compound (VIII), wherein R₂₁ is an acyl moiety corresponding to the acylating agent. The reaction mixture optionally contains an acid scavenger.

In one embodiment, the reaction mixture comprises about 1 molar equivalent to about 10 molar equivalents of the acylating agent to 1 molar equivalent of compound (VII). In another example, the reaction mixture is charged with about 1 molar equivalent to about 3 molar equivalents of the acylating agent to 1 molar equivalent of compound (VII).

In one embodiment, the reaction between the acylating agent and compound (VII) occurs in the presence of an acid scavenger, wherein the reaction mixture comprises about 1 molar equivalent to about 3 molar equivalents of the acid scavenger.

The temperature of the reaction mixture ranges from about −10° C. to about 75° C.

In another example, the reaction temperature ranges from about −10° C. to about 65° C. In still another example, the reaction temperature ranges from about 35° C. to about 65° C. The reaction mixture is permitted to react up to a couple of days. In one example, the reaction is carried out from about 1 hour to about 24 hours. In another example, the reaction time is from about 2 hours to about 16 hours. In another example, the reaction time is from about 2 hours to about 8 hours.

In one embodiment, R₂₁ is —CO—R₂₂, wherein R₂₂ is hydrocarbyl or substituted hydrocarbyl. In another example, the acylating agent is an acid halide is a C₁-C₁₈ acid halide selected from alkyl acid halides and alkoxy-alkyl halides. Examples of acylating agents include, but are not limited to, acetyl chloride, ethanoyl chloride, propionyl chloride, propionic anhydride, methyl ketene, butanoyl chloride, alkyl acid cyanides, and the like. In one embodiment, the alkyl group contains between 1 to 18 carbons. In another embodiment, the alkyl group contains between 2 to 4 carbons.

The solvent contained in the reaction mixture can be any solvent that is inert to the reaction occurring in Step 4. Examples of such solvents include, but are not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; lower alkanol such as methanol, ethanol, isopropanol, 1-butanol, tert-butanol, and the like; ketones such as 4-methyl-2-pentanone and the like; ethers such as 1,4-dioxane, tetrahydrofuran (THF), 1,1-oxybisethane, and the like; nitrobenzene; and mixtures thereof. In one example, the reaction mixture contains acetonitrile.

The acid scavenger can include metal hydrides, hydroxides, carbonates, bicarbonates, amines, and the like.

In one embodiment, the reaction mixture can also comprise an acid catalyst. The acid catalyst can include any Lewis acid, for example, aluminum chloride, boron trifluoride, sulfuric acid, hydrochloric acid, phosphoric acid, and the like. In one embodiment, the acid concentration is between about 1% to about 30%. In another embodiment, the acid concentration is between about 10% to about 20%. In another embodiment, the acid concentration is about 10%.

After the reaction is completed, water and a base are added to the reaction mixture to adjust the pH above 7. Solvent extraction is conducted with an organic solvent. The solvent is removed to obtain the crude product. Compound (VIII) may be isolated from the crude product through chromatography or distillation. Alternatively, the salt form of the crude product may be isolated through recrystallization by protonation with an acid.

The overall process of the present invention for synthesizing opiate or opioid analgesics and anesthetic that incorporates the individual steps described above is illustrated in Scheme 5, below.

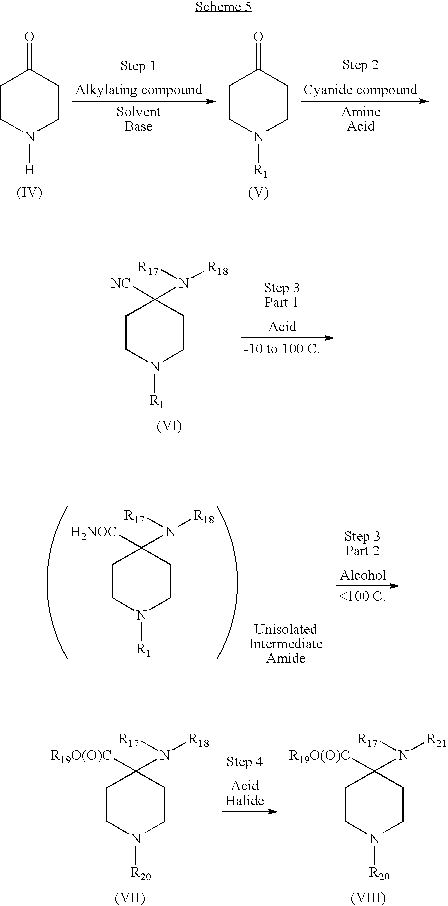

The process of the present invention described above significantly improves the synthesis reactions for producing analgesics by reducing a series of three reaction steps as described in detail in U.S. Pat. No. 5,106,983, to a single two-part reaction, identified above as Step 3, taking place in a single reaction mixture wherein no product is isolated between the parts. The reaction process is used to hydrolyze and esterify intermediates of analgesics. In the process of synthesizing remifentanil, the reaction is illustrated in Scheme 6.

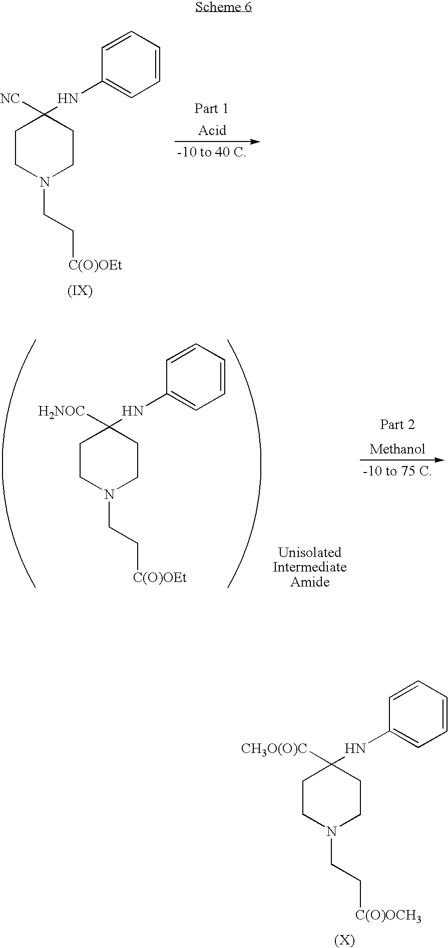

In Part 1 of Scheme 6, compound (IX) is hydrolyzed in acid to form an intermediate amide in situ. In Part 2 of Scheme 6, methanol is added to the reaction mixture of Part 1 of Scheme 2. The intermediate amide is esterified to form compound (X), wherein the amide moiety is esterified into a methyl ester and the ethyl ester is transesterified into a methyl ester. The other reaction conditions for the reaction of Scheme 6 are the same as described in detail above for the reaction of Scheme 3.

Similarly, a single two-part reaction as illustrated in Scheme 7 can be used to synthesize intermediates in the process of synthesizing carfentanil.

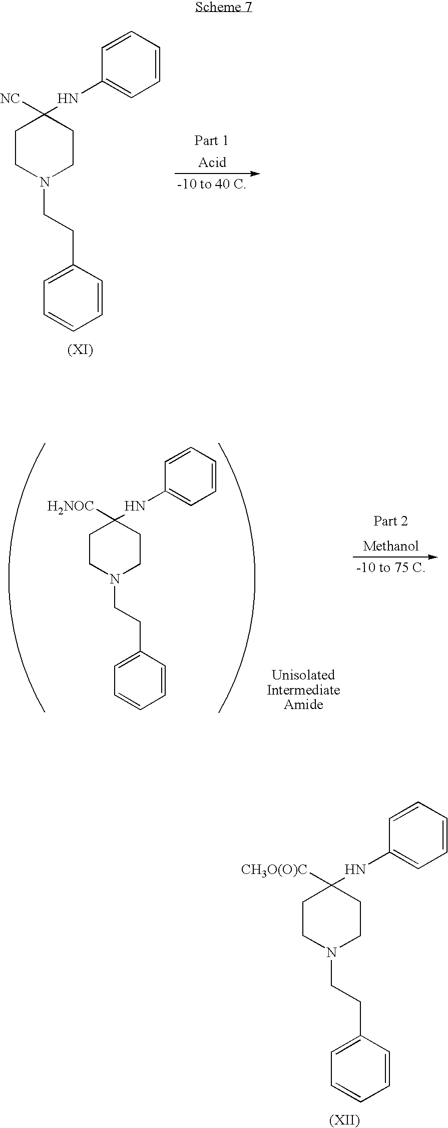

In Part 1 of Scheme 7, compound (XI) is hydrolyzed in acid to form an intermediate amide in situ. In Part 2 of Scheme 7, methanol is added to the reaction mixture of Part 1 of Scheme 2. The intermediate amide is esterified to form compound (XII), wherein the amide moiety is esterified into a methyl ester and the ethyl ester is transesterified into a methyl ester. The other reaction conditions for the reaction of Scheme 7 are the same as described in detail above for the reaction of Scheme 3.

In one embodiment of the present invention, a process for synthesizing remifentanil is provided. An illustration of this process is illustrated below in Scheme 8.

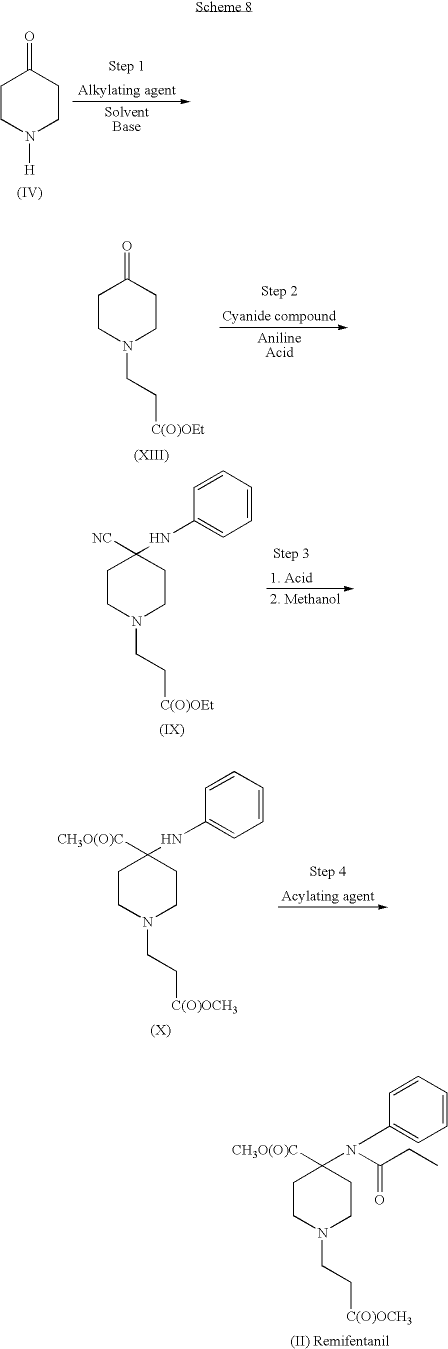

An acid salt of compound (IV), for example 4-piperidone hydrochloride, is reacted with an alkylating agent in Step 1 in the presence of a solvent and an acid scavenger to form intermediate compound (XIII). The alkylating agent is selected from the group consisting of alkyl acrylate, acrylic acid, acryronitrile, acrylamide, and acrolein.

In one embodiment, the reaction mixture comprises about 1 molar equivalent to about 3 molar equivalents of the alkylating agent and about 1 molar equivalent to about 3 molar equivalents of the acid scavenger (i.e., a base) to 1 molar equivalent of compound (IV). The solvent to compound (IV) ratio on a wt. basis is about 1:10 to 1:100.

In another example, the reaction mixture is charged with about 1 molar equivalent to about 1.5 molar equivalents of an alkylating agent and about 1 molar equivalent to about 1.5 molar equivalents of an acid scavenger to 1 molar equivalent of 4-piperidine hydrochloride.

The temperature of the reaction mixture during the reaction ranges from about −10° C. to about 65° C. In another example, the reaction temperature ranges from about 10° C. to about 40° C. The reaction mixture is permitted to react up to a couple of days. In one embodiment, the reaction is carried out up to about 24 hours. In another example, the reaction time is from about 2 hours to about 6 hours.

The reaction mixture contains a base to neutralize the acid salt of compound (IV) is the reaction mixture also comprises a base to neutralize the acid salt of compound (IV). Examples of the base include sodium hydroxide, potassium hydroxide, metal alkoxides, metal hydrides, metals, amines, quaternary alkyl ammonia hydroxides, and any other base in that can neutralize an acid salt of compound (IV). Examples of metal alkoxides and metal hydrides include sodium, potassium, cesium, magnesium, aluminum alkoxides and hydrides and the like. Examples of metals include scavenging metals such as sodium, potassium, magnesium, and the like.

The solvent used in the reaction mixture can include water and/or one or more organic solvents. Examples of organic solvents include, but are not limited to acetonitrile, acetone, dichloromethane, chloroform, tetrahydrofuran, n,n-dimethylformamide, dimethylsulfoxide, ethylacetate, and the like.

Compound (XIII) can be isolated by utilizing isolation procedures known in the art such as those described for the above schemes.

In Step 2, compound (XIII) is reacted in a reaction mixture with a cyanide compound and aniline in the presence of an acid to form compound (IX).

In one embodiment, the reaction mixture comprises about 1 molar equivalent to about 3 molar equivalents of aniline and about 1 molar equivalent to about 3 molar equivalents of the cyanide compound to 1 molar equivalent of compound (XIII). The acidic medium to compound (XIII) ratio on a wt. basis is about 1:10 to 1:100.

In another embodiment, the reaction mixture is charged with about 1 equivalent to about 1.2 equivalents of the aniline and about 1 equivalent to about 1.2 equivalents of the cyanide compound in a w/w ratio of about 10 to about 20 of an acidic medium.

The temperature of the reaction mixture ranges from about −10° C. to about 65° C. In another example, the reaction temperature ranges from about 10° C. to about 40° C. The reaction mixture is permitted to react up to a couple of days. In one example, the reaction is carried out up to about 24 hours. In another example, the reaction time is from about 2 hours to about 6 hours.

Non-limiting examples of cyanide compounds include sodium cyanide, potassium cyanide, trimethylsilyl cyanide, hydrogen cyanide, and the like.

The acid may include any organic or inorganic acid to adjust the pH below about 7. Non-limiting examples of acids include acetic acid, hydrochloric acid, sulfuric acid, phosphoric acid, oxalic acid, and the like. In one embodiment, acetic acid is utilized to adjust the reaction mixture pH to below about 7.

The reaction can be conducted from the presence or absence of water. If the reaction takes place under anhydrous conditions, excess amount of a solvent is used in the reaction mixture. In one embodiment, the solvent is comprised of organic solvents including, but not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; alcohols having one or more carbons such as methanol, ethanol, isopropanol, 1-butanol, tert-butanol, and the like; ketones such as 4-methyl-2-pentanone and the like; ethers such as 1,4-dioxane, tetrahydrofuran (THF), 1,1-oxybisethane, and the like; nitrobenzene; and mixtures thereof.

In another embodiment, the solvent can contain between about 10% to about 100% acid. In one embodiment, the reaction mixture can contain between about 0% to about 90% water.

Compound (IX) can be isolated by utilizing isolation procedures known in the art such as those described for the above schemes.

Step 3 is a two-part reaction taking place in a single reaction mixture wherein no product is isolated between the parts. In Part 1 of Step 3, compound (IX) is hydrolyzed in acid to form an intermediate amide in situ. The reaction mixture can optionally comprise a solvent.

In one embodiment, the reaction mixture comprises about 3 molar equivalents to about 10 molar equivalents of the acid to 1 molar equivalent of compound (IX). In another example, the reaction mixture comprises about 3 molar equivalents to about 5 molar equivalents of the acid to 1 molar equivalent of compound (IX).

The reaction mixture temperature is from about −10° C. to about 40° C. In another example, the reaction mixture temperature is from about 15° C. to about 35° C. In still another example, the reaction mixture temperature is from about 10° C. to about 30° C. The reaction mixture is permitted to react up to a couple of days. In one example, the reaction is carried out up to about 24 hours. In another example, the reaction time is from about 2 hours to about 8 hours.

The acid source can be an organic or inorganic acid to adjust the pH of the reaction mixture below about 7. In one embodiment, the acid is selected from acetic acid, hydrochloric acid, sulfuric acid, methansulfonic acid, phosphoric acid, oxalic acid, and the like. In one example, the acid concentration is between 10% and about 99%, preferably between 70% and about 99%, with the balance comprising water. In still another example, the acid is selected from sulfuric acid or methansulfonic acid.

In one embodiment, the reaction mixture contains a solvent selected from the organic solvents described above for Scheme 2. In one embodiment, the solvent comprises between about 10% to about 99% solvent.

If the reaction takes place under anhydrous conditions, excess amount of alcohol is used as a solvent in the reaction mixture. In one embodiment, the alcohol is an aliphatic alcohol having 1 to 3 carbons.

In Part 2 of Step 3, methanol is added to the reaction mixture of Part 1 of Step 3. The intermediate amide is esterified to form compound (X), wherein the amide moiety is esterified into a methyl ester and the ethyl ester is transesterified into a methyl ester.

In one embodiment, about 10 parts to about 50 parts of methanol are added to the reaction mixture of Part 2 of Scheme 2. In another example, about 10 to about 20 parts of alcohol are added to the reaction mixture of Part 2 of Scheme 2.

The reaction mixture temperature is from about −10° C. to about 75° C. In another example, the reaction mixture temperature is from about 40° C. to about 65° C. The reaction mixture is permitted to react for about 24 hours to about 150 hours. In another example, the reaction time is from about 60 hours to about 100 hours.

Compound (X) can be isolated by utilizing isolation procedures known in the art such as those described for the above schemes.

In Step 4, compound (X) is reacted with an acylating agent in a reaction mixture containing a solvent to form compound (II). The reaction mixture optionally contains an acid scavenger.

In one embodiment, the reaction mixture comprises about 1 molar equivalent to about 10 molar equivalents of the acylating agent to 1 molar equivalent of compound (X). In another example, the reaction mixture is charged with about 1 molar equivalent to about 3 molar equivalents of the acylating agent to 1 molar equivalent of compound (X).

In one embodiment, the reaction between the acylating agent and compound (X) occurs in the presence of an acid scavenger, wherein the reaction mixture comprises about 1 molar equivalent to about 3 molar equivalents of the acid scavenger.

The temperature of the reaction mixture ranges from about −10° C. to about 75° C. In another example, the reaction temperature ranges from about −10° C. to about 65° C. In still another example, the reaction temperature ranges from about 35° C. to about 65° C. The reaction mixture is permitted to react up to a couple of days. In one embodiment, the reaction is carried out from about 1 hour to about 24 hours. In another example, the reaction time is from about 2 hours to about 16 hours. In another example, the reaction time is from about 2 hours to about 8 hours.

In one embodiment the acylating agent is selected from propionyl halide or propionic anhydride. In another example, the acylating agent comprises propionyl chloride.

The solvent contained in the reaction mixture can be any solvent that is inert to the reaction occurring in Step 4. Examples of such solvents include, but are not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; lower alkanol such as methanol, ethanol, 1-butanol, and the like; ketones such as 4-methyl-2-pentanone and the like; ethers such as 1,4-dioxane, tetrahydrofuran (THF), 1,1-oxybisethane, and the like; nitrobenzene; and mixtures thereof. In one example, the reaction mixture contains acetonitrile.

The acid scavenger can include metal hydrides, hydroxides, carbonates, bicarbonates, amines, and the like.

In one embodiment, the reaction mixture can also comprise an acid catalyst. The acid catalyst can include any Lewis acid, for example, aluminum chloride, boron trifluoride, sulfuric acid, hydrochloric acid, phosphoric acid, and the like. In one embodiment, the acid concentration is between about 1% to about 30%. In another embodiment, the acid concentration is between about 10% to about 20%. In another embodiment, the acid concentration is about 10%.

After the reaction is completed, water and a base are added to the reaction mixture to adjust the pH above 7. Solvent extraction is conducted with an organic solvent. The solvent is removed to obtain the crude product. Compound (II) may be isolated from the crude product through chromatography or distillation. Alternatively, the salt form of the crude product may be isolated through recrystallization by protonation with an acid.

In another embodiment of the present invention, a process for synthesizing carfentanil, compound (III) is provided. An illustration of this process is illustrated below in Scheme 9.

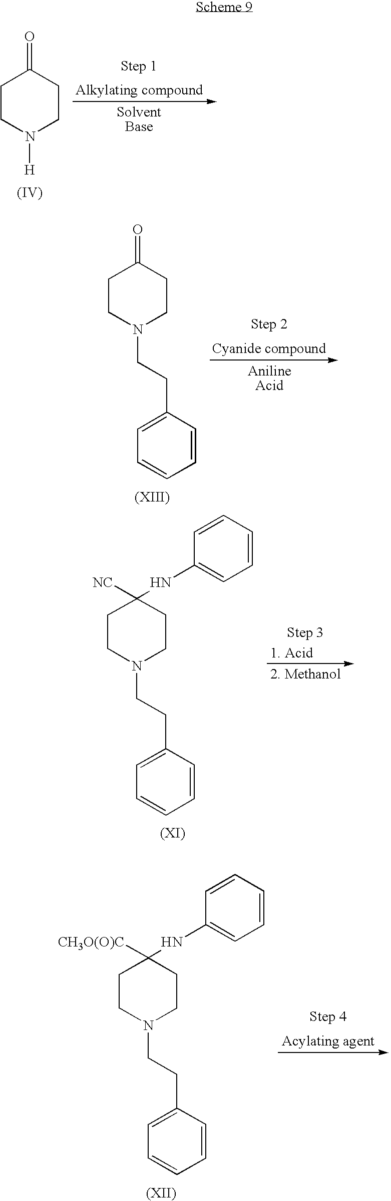

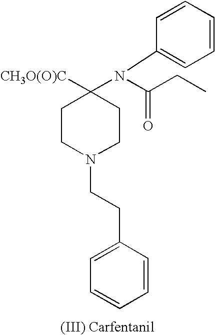

An acid salt of compound (IV), for example 4-piperidone hydrochloride, is reacted with an alkylating compound in Step 1 in the presence of a solvent and a base to form intermediate compound (XIII). Examples of an alkylating agent include any electrophile containing phenylethyl group, such as a phenylethyl halide, toluene sulfonate, methane sulfonate, and the like.

Alternatively, in lieu of synthesizing compound (XIII) from compound (IV), 1-(2-phenylethyl)-4-piperidone, compound (XIII), may be obtained from a vendor as a starting reactant wherein the process for synthesizing carfentanil would begin at Step 2 of Scheme 5.

The other reaction conditions for the reaction of Scheme 9 are the same as described in detail above for the reaction of Scheme 8.

After the reaction is completed, water and a base are added to the reaction mixture to adjust the pH above 7. Solvent extraction is conducted with an organic solvent. The solvent is removed to obtain the crude product. Compound (III) may be isolated from the crude product through chromatography or distillation. Alternatively, the salt form of the crude product may be isolated through recrystallization by protonation with an acid.

The processes of the present invention are useful in the synthesis of intermediate compounds that can be utilized in the preparation of opiate or opioid analgesics or anesthetics.

The product compounds synthesized according to the process of the present invention may be used as synthetic opiates or opioids for analgesic or anesthetic purposes. In particular, the remifentanil compounds of the present invention can be used as anesthetics in surgical procedures wherein the compounds have a beneficially short half-life in humans that permit patients to awaken shortly after a surgical procedure has been concluded.

### ABBREVIATIONS AND DEFINITIONS

The term “acyl” is a radical provided by the residue after removal of hydroxyl from an organic acid, for example, COOH of an organic carboxylic acid, e.g., RC(O)—, wherein R is R₂₄, R₂₄O—, R₂₄R₂₅N—, or R₂₅S—, R₂₄ is hydrocarbyl, heterosubstituted hydrocarbyl, or heterocyclo and R₂₅ is hydrogen, hydrocarbyl or substituted hydrocarbyl. Examples of such acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, propenyl, butenyl and 4-methylbutenyl. The terms “alkenyl” and “lower alkenyl” also are radicals having “cis” and “trans” orientations, or alternatively, “E” and “Z” orientations. The term “cycloalkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “amino” as used herein alone or as part of another group denotes the moiety —NR₂₆R₂₇ wherein R₂₆ and R₂₇ are hydrocarbyl, substituted hydrocarbyl or heterocyclo.

The terms “halide,” “halogen,” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, and preferably 5 or 6 atoms in each ring. The heterocyclo group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties comprise 1 to 18 carbon atoms. They may be straight or branched chain or cyclic and include methyl, ethyl, propyl, isopropyl, allyl, benzyl, hexyl and the like.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxy, alkynoxy, aryloxy, hydroxy, keto, acyl, acyloxy, nitro, tertiaryamino, amido, nitro, cyano, ketals, acetals, esters and ethers.

The following examples are provided in order to more fully illustrate the present invention.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained.

As various changes could be made in the above methods and products without departing from the scope of the invention, it is intended that all matter contained in the above description and shown in any accompanying drawings shall be interpreted as illustrative and not in a limiting sense.

1. A process for the preparation of an analgesic or anesthetic, comprising:
reacting a compound (IV) having the formula: 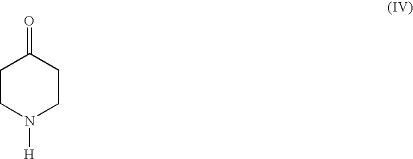
with an alkylating compound in the presence of a solvent to form intermediate compound (V): 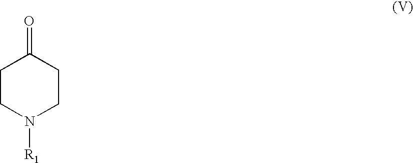
wherein R₁ is a hydrocarbyl or substituted hydrocarbyl;
reacting the intermediate compound (v) with an amine and a cyanide compound, in the presence of a first acid to form an intermediate compound (VI): 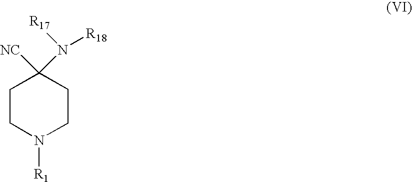
wherein R₁₇ and R₁₁ are independently selected from the group comprising hydrogen, hydrocarbyl, and substituted hydrocarbyl, reacting the intermediate compound (VI) with a second acid to form an intermediate amide;
reacting the intermediate amide with an alcohol, R₁₉OH, to form an intermediate compound (VII): 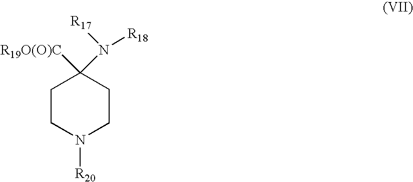
wherein
R₁₉ is hydrocarbyl or substituted hydrocarbyl; and
R₂₀ is hydrocarbyl or substituted hydrocarbyl; and
reacting the intermediate compound (VII) with an acylating agent to form a compound (VIII) having the formula: 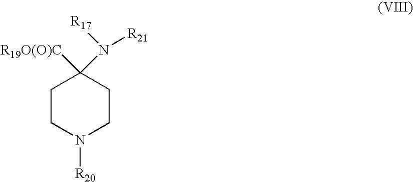
wherein R₂₁ is <(O)—R₂₂, wherein R₂₂ is hydrocarbyl or substituted hydrocarbyl.

2. The process of claim 1, wherein the solvent is water, an organic solvent, or a mixture thereof.

3. The process of claim 1, wherein R₁ is selected from the group consisting of R₅OC(O)R₆—, R₇C(O)OR₈—, R₉OR₁₀OC(O)R₁₁—, R₁₂R₁₃—, and R₁₄R₁₅—,
wherein
R₅, R₆, R₇, R₈, R₉, R₁₀, R₁₁, R₁₃, and R₁₅ are hydrocarbyl or substituted hydrocarbyl;
R₁₂ is cycloalkyl; and
R₁₄ is a heterocyclic comprising 1 to 5 hetero-atoms.

4. The process of claim 1, wherein R₁₉ and R₂₀ are independently selected from the group comprising hydrogen, alkyl, alkoxyalkyl, aryl, substituted aryl, and 5- to 7-member cycloalkyl or heterocyclic structures.

5. The process of claim 1, wherein R₂₂ is selected from the group consisting of R₅OC(O)R₆—, R₇C(O)OR₈—, R₉R₁₀OC(O)R₁₁—, R₁₂R₁₃—, and R₁₄R₁₅—,
wherein
R₅, R₆, R₇, R₈, R₉, R₁₀, R₁₁, R₁₃, and R₁₅ are hydrocarbyl or substituted hydrocarbyl;
R₁₂ is cycloalkyl; and
R₁₄ is a heterocyclic comprising 1 to 5 hetero-atoms.

6. The process of claim 1, wherein R₂₁ is selected from the group consisting of —CO—R₂₂, wherein R₂₂ is hydrocarbyl or substituted hydrocarbyl.

7. The process of claim 1, wherein the alkylating compound is selected from the group consisting of methyl acrylate, ethyl acrylate, acrylic acid, acrylonitrile, acrylamide, acrolein, phenylethyl halide, tolylate, mesoilate, styrene, and substituted styrene.

8. The process of claim 1, wherein the organic solvent is selected from the group consisting of acetonitrile, acetone, dichloromethane, chloroform, n,n-dimethylformamide, dimethylsulfoxide, ethylacetate, dichloroethane, aromatic hydrocarbons, benzene, toluene, xylene, methanol, ethanol, 1-butanol, 4-methyl-2-pentanone, tetrahydrofuran, 1,4-dioxane, 1,1-oxybisethane, nitrobenzene; and mixtures thereof.

9. The process of claim 1, wherein the cyanide compound comprises sodium cyanide, potassium cyanide, trimethylsilyl cyanide, or hydrogen cyanide.

10. The process of claim 1, wherein the amine is selected from the group consisting of aniline; substituted phenyl amine compounds wherein the substituted constituents include C₁-C₁₈ hydrocarbyl or substituted hydrocarbyl groups.

11. The process of claim 1, wherein the intermediate compound (V) is reacted with aniline in the presence of an acid, wherein the acid is selected from the group consisting of acetic acid, hydrochloric acid, sulfuric acid, methansulfonic acid, phosphoric acid, and oxalic acid.

12. The process of claim 1, wherein the intermediate compound (VI) is reacted with an acid wherein the acid is selected from the group consisting of acetic acid, hydrochloric acid, sulfuric acid, methansulfonic acid, phosphoric acid oxalic acid, to form the intermediate amide.

13. The process of claim 1, wherein the alcohol comprises a C₁-C₁₈ aliphatic alcohol.

14. The process of claim 13, wherein the alcohol comprises methanol, ethanol, propanol, isopropanol, butanol, tert-butanol, sec-butanol, pentanol, or hexanol.

15. The process of claim 1, wherein the acylating agent is selected from the group consisting of ethanoyl chloride, propionyl chloride, propionic anhydride, methyl ketene, butanoyl chloride, and alkyl acid cyanides.

16. The process of claim 1, wherein the intermediate compound (VII) is reacted with an acylating agent in a reaction mixture in the presence of an acid scavenger.

17. The process of claim 16, wherein the acid scavenger is selected from the group consisting of metal hydrides, hydroxides, carbonates, bicarbonates, and amines.

18. The process of claim 16, wherein the reaction mixture further comprises a solvent wherein the solvent is selected from the group comprising acetonitrile; acetone; dichloromethane; chloroform; n,n-dimethylformamide; dimethylsulfoxide; ethylacetate; dichloroethane; benzene; toluene; xylene; methanol; ethanol; isopropanol, 1-butanol; tert-butanol; 4-methyl-2-pentanone; 1,4-dioxane, tetrahydrofuran; 1,1-oxybisethane, nitrobenzene; and mixtures thereof.

19. The process of claim 1, wherein the compound (VIII) is remifentanil or carfentanil.

20. A process of synthesizing an intermediate of opiate or opioid analgesics or anesthetics, the process comprising:
reacting compound (IV) having the formula: 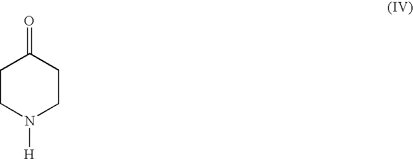
with an alkylating agent, a solvent, and a base to form an intermediate compound (V) having the formula: 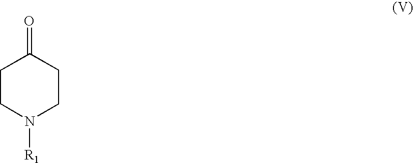
wherein R₁ is hydrocarbyl or substituted hydrocarbyl.

21. The process of claim 20, wherein R₁ is selected from the group consisting of R₅OC(O)R₆—, R₇C(O)OR₈—, R₉OR₁₀OC(O)R₁₁—, R₁₂R₁₃—, and R₁₄R₁₅—,
wherein
R₅, R₆, R₇, R₈, R₉, R₁₀, R₁₁, R₁₃, and R₁₅ are hydrocarbyl or substituted hydrocarbyl;
R₁₂ is cycloalkyl; and
R₁₄ is a heterocyclic comprising 1 to 5 hetero-atoms.

22. A process of synthesizing an intermediate of opiate or opioid analgesics or anesthetics, the process comprising:
reacting intermediate compound (V) having the formula: 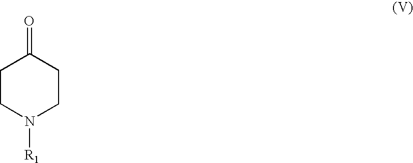
with cyanide compound, an amine, and an acid to form an intermediate compound (VI) having the formula: 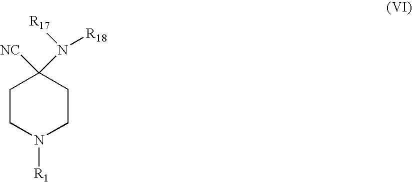
wherein
R₁ is a hydrocarbyl or substituted hydrocarbyl; and
R₁₇ and R₁₉ are independently selected from hydrogen, hydrocarbyl, or substituted hydrocarbyl.

23. The process of claim 22, wherein R₁₇ and R₁₈ are independently selected from hydrogen, alkyl, alkoxyalkyl, aryl with and without substitution, and a hydrocarbyl or substituted hydrocarbyl 5- to 7-member cyclic structure.

24. A process of synthesizing an intermediate of opiate or opioid analgesics or anesthetics, the process comprising:
reacting intermediate compound (VI) having the formula: 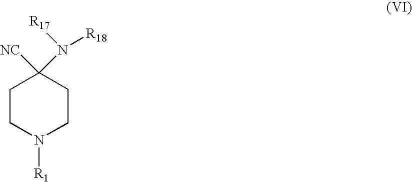
with an acid and an alcohol, R₁₉OH, in a reaction mixture to form an intermediate compound (VII) having the formula: 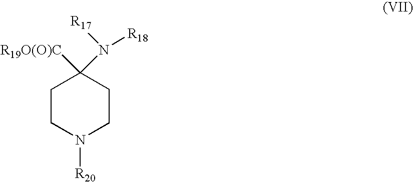
wherein
R₁ is a hydrocarbyl or substituted hydrocarbyl;
R₁₇ and R₁₈ are independently selected from hydrogen, hydrocarbyl, or substituted hydrocarbyl;
R₁₉ is hydrocarbyl or substituted hydrocarbyl; and
R₂₀ is hydrocarbyl or substituted hydrocarbyl.

25. The process of claim 24, wherein R₂₂ is a group selected from R₅OC(O)R₆—, R₇C(O)OR₈—, R₉OR₁₀OC(O)R₁₁—, R₁₂R₁₃—, and R₁₄R₁₅—,
wherein
R₅, R₆, R₇, R₈, R₉, R₁₀, R₁₁, R₁₃, and R₁₅ are hydrocarbyl or substituted hydrocarbyl;
R₁₂ is cycloalkyl; and
R₁₄ is a heterocyclic comprising 1 to 5 hetero-atoms.

26. The process of claim 25, wherein the heteroatoms are selected from the group consisting of oxygen, sulfur, and nitrogen.

27. The process of claim 24, wherein intermediate compound (VI) has the structure:

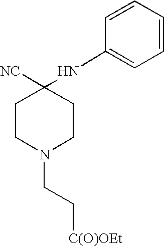

28. The process of claim 24, wherein the alcohol comprises methanol and wherein the intermediate compound (VII) has the formula:

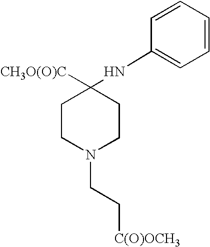

29. The process of claim 24, wherein intermediate compound (VI) has the structure:

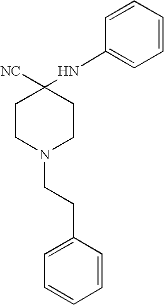

30. The process of claim 24, wherein the alcohol comprises methanol and intermediate compound (VII) having the formula:

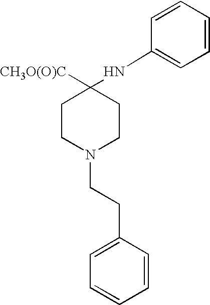

31. The process of claim 24, wherein the reaction takes place in a single reaction mixture.

32. The process of claim 24, wherein the intermediate compound (VI) is reacted with acid in the reaction mixture at a temperature from about −10° C. to about 40° C.

33. The process of claim 24, wherein the intermediate amide is reacted with methanol in the reaction mixture at a temperature from about −10° C. to about 75° C.

34. The process of claim 24, wherein the intermediate amide is reacted with methanol for up to 200 hours.
